(19) United States
(12) Patent Application Publication
(10) Pub. No.: US 2021/0130309 A1
(43) Pub. Date: May 6, 2021

(54) PROCESS FOR THE PRODUCTION OF ETHYLENE OXIDE

(71) Applicant: SHELL OIL COMPANY, HOUSTON, TX (US)

(72) Inventors: Guus VAN ROSSUM, Amsterdam (NL); Ivana Daniela ESPOSITO CASSIBBA, Amsterdam (NL); Ronald Jan SCHOONEBEEK, Amsterdam (NL); Alouisius Nicolaas Renée BOS, Amsterdam (NL); Peter Alexander SCHUT, Amsterdam (NL); Laura Mariel CALVO, Amsterdam (NL)

(21) Appl. No.: 17/045,807

(22) PCT Filed: Apr. 4, 2019
(86) PCT No.: PCT/EP2019/058446

§ 371 Date: Oct. 7, 2020

(30) Foreign Application Priority Data
Apr. 9, 2018 (EP) 18166282.6

(51) Int. Cl. C07D 301/06; C07C 5/48; C07C 29/10

(52) U.S. Cl. CPC C07D 301/06; C07C 11/04; C07C 29/106; C07C 5/48

(57) ABSTRACT

The invention relates to a Process for the production of ethylene oxide, comprising the steps of: (a) producing ethylene by subjecting a stream comprising ethane to oxidative dehydrogenation conditions, resulting in a stream comprising ethylene, ethane, water and acetic acid; (b) separating at least part of the stream resulting from step (a) into a stream comprising ethylene and ethane and a stream comprising water and acetic acid; (c) producing ethylene oxide by subjecting ethylene and ethane from the stream comprising ethylene and ethane resulting from step (b) to oxidation conditions, resulting in a stream comprising ethylene oxide, ethylene, ethane and water; (d) separating at least part of the stream resulting from step (c) into a stream comprising ethylene and ethane and a stream comprising ethylene oxide and water; (e) recycling ethylene and ethane from the stream comprising ethylene and ethane resulting from step (d) to step (a), wherein carbon dioxide is produced in steps (a) and (c) and is removed in an additional step between steps (b) and (c) and/or between steps (d) and (e).

### FIELD OF THE INVENTION

The present invention relates to a process for the production of ethylene oxide.

### BACKGROUND OF THE INVENTION

Ethylene oxide is used as a chemical intermediate, primarily for the production of ethylene glycols but also for the production of ethoxylates, ethanol-amines, solvents and glycol ethers. It may be produced by the direct oxidation of ethylene. Several processes for producing the ethylene starting material are known. For example, it is known to produce ethylene by oxidative dehydrogenation (oxydehydrogenation; ODH) of ethane. Said ethane ODH and ethylene oxide production processes have in common that oxygen is used.

WO2012101069 discloses a process wherein the above-mentioned ethane ODH and ethylene oxide production processes are combined. WO2012101069 discloses a process for the production of ethylene oxide, comprising the steps of: producing ethylene by subjecting a stream comprising ethane to oxydehydrogenation conditions resulting in a stream comprising ethylene and unconverted ethane; producing ethylene oxide by subjecting ethylene and unconverted ethane from the stream comprising ethylene and unconverted ethane to oxidation conditions resulting in a stream comprising ethylene oxide, unconverted ethylene and unconverted ethane; and recovering ethylene oxide from the stream comprising ethylene oxide, unconverted ethylene and unconverted ethane.

Further, in a particular embodiment of WO2012101069, a stream comprising unconverted ethylene and unconverted ethane is separated from the above-mentioned stream comprising ethylene oxide, unconverted ethylene and unconverted ethane, the stream comprising unconverted ethylene and unconverted ethane is separated into a stream comprising unconverted ethylene which is recycled to the step of producing ethylene oxide and a stream comprising unconverted ethane which is recycled to the step of producing ethylene. That is to say, in said embodiment of WO2012101069, unconverted ethylene is recycled to the ethylene oxide production step and unconverted ethane is recycled to the ethane ODH step.

The above-mentioned embodiment is illustrated in FIG. 3 of WO2012101069. In said FIG. 3, stream 11 comprising unconverted ethylene and ethane is split into two substreams 11a and 11b. Substream 11a is recycled to ethylene oxide production unit 5. Substream 11b is fed to ethylene/ethane separation unit 12. Stream 13 comprising unconverted ethylene and stream 14 comprising unconverted ethane are recycled to ethylene oxide production unit 5 and to ethylene production unit 2, respectively. Further, as disclosed in WO2012101069, a third stream may be separated in ethylene/ethane separation unit 12, namely a top bleed (purge) stream comprising uncondensable components, such as oxygen and/or argon.

It is an object of the present invention to provide a simplified integrated process for the production of ethylene oxide from ethane, involving ethane ODH followed by ethylene oxidation, which may be a technically advantageous, efficient and affordable process. Such technically advantageous process would preferably result in a lower energy demand and/or lower capital expenditure.

### SUMMARY OF THE INVENTION

Surprisingly it was found that the above-mentioned object may be achieved by an integrated process combining an ethane ODH step and a subsequent ethylene oxide production step, wherein both ethylene and ethane from the stream comprising ethylene oxide, ethylene, ethane and water resulting from the ethylene oxide production step are recycled to the ethane ODH step. For example, it has been found that with the present fully integrated process, risks involving the use of an oxidizing agent (e.g. oxygen), which is needed in both the ethane ODH step and the ethylene oxide production step, may be reduced. For a further explanation and for a discussion of other advantages, reference is made to the below section on “Advantages of the present invention”.

Accordingly, the present invention relates to a process for the production of ethylene oxide, comprising the steps of:

(a) producing ethylene by subjecting a stream comprising ethane to oxidative dehydrogenation conditions, resulting in a stream comprising ethylene, ethane, water and acetic acid;

(b) separating at least part of the stream resulting from step (a) into a stream comprising ethylene and ethane and a stream comprising water and acetic acid;

(c) producing ethylene oxide by subjecting ethylene and ethane from the stream comprising ethylene and ethane resulting from step (b) to oxidation conditions, resulting in a stream comprising ethylene oxide, ethylene, ethane and water;

(d) separating at least part of the stream resulting from step (c) into a stream comprising ethylene and ethane and a stream comprising ethylene oxide and water;

(e) recycling ethylene and ethane from the stream comprising ethylene and ethane resulting from step (d) to step (a),

wherein carbon dioxide is produced in steps (a) and (c) and is removed in an additional step between steps (b) and (c) and/or between steps (d) and (e).

Further, the present invention relates to a process for the production of monoethylene glycol, wherein at least part of the ethylene oxide obtained in the above-mentioned process is converted to monoethylene glycol.

### BRIEF DESCRIPTION OF THE DRAWING

FIG. 1 shows an embodiment of the present invention.

### DETAILED DESCRIPTION OF THE INVENTION

The process of the present invention comprises steps (a) to (e). Said process may comprise one or more intermediate steps between steps (a) and (b), between steps (b) and (c), between steps (c) and (d), and between steps (d) and (e). Further, said process may comprise one or more additional steps preceding step (a) and/or following step (e).

While the process of the present invention and a composition or stream used in said process are described in terms of “comprising”, “containing” or “including” one or more various described steps and components, respectively, they can also “consist essentially of” or “consist of” said one or more various described steps and components, respectively.

In the context of the present invention, in a case where a composition or stream comprises two or more components, these components are to be selected in an overall amount not to exceed 100 vol. % or 100 wt. %.

Within the present specification, “substantially no” means that no detectible amount of the component in question is present in the composition or stream.

Further, within the present specification, by “fresh ethane” reference is made to ethane which does not comprise unconverted ethane. Within the present specification, by “unconverted ethane” reference is made to ethane that was subjected to oxidative dehydrogenation conditions in step (a) of the process of the present invention, but which was not converted. Further, within the present specification, by “unconverted ethylene” reference is made to ethylene that was subjected to oxidation conditions in step (c) of the process of the present invention, but which was not converted.

Step (a)

Step (a) of the present process comprises producing ethylene by subjecting a stream comprising ethane to oxidative dehydrogenation conditions, resulting in a stream comprising ethylene, ethane, water and acetic acid. This step is also referred to as the ethane ODH step. Since in step (e) of the present process ethylene and ethane are recycled to step (a), in step (a) a stream comprising ethylene and ethane is subjected to oxidative dehydrogenation conditions, resulting in a stream comprising ethylene, ethane, water and acetic acid. Step (a) may comprise contacting the stream comprising ethylene and ethane with oxygen (O₂). Further, said contacting may be carried out in the presence of a catalyst comprising a mixed metal oxide. Such catalyst is further described below.

In ethane ODH step (a), ethylene is produced by oxidative dehydrogenation of ethane. In step (a), part of the ethylene as formed in step (a) and the ethylene as recycled in step (e) to step (a) are oxidized into acetic acid. In step (a), ethylene may also be dehydrogenated into acetylene (ethyne). Ethane may also be directly converted into acetic acid or acetylene. In step (a), carbon dioxide (CO₂) and carbon monoxide (CO) are produced, for example by combustion of ethane and/or ethylene and/or acetic acid and/or acetylene.

In ethane ODH step (a), ethane, ethylene and oxygen (O₂) may be fed to a reactor. Said components may be fed to the reactor together or separately. That is to say, one or more feed streams, suitably gas streams, comprising one or more of said components may be fed to the reactor. For example, one feed stream comprising oxygen, ethane and ethylene may be fed to the reactor. Alternatively, two or more feed streams, suitably gas streams, may be fed to the reactor, which feed streams may form a combined stream inside the reactor. For example, one feed stream comprising oxygen, another feed stream comprising fresh ethane and still another feed stream comprising unconverted ethane and unconverted ethylene, which latter stream is recycled in step (e) to step (a) of the present process, may be fed to the reactor separately. In ethane ODH step (a), ethane, ethylene and oxygen are suitably fed to a reactor in the gas phase.

In the present invention, the weight ratio of ethylene to ethane as fed to ethane ODH step (a) may be in the range of from 0.1:1 to 2:1, preferably of from 0.2:1 to 1.5:1, more preferably of from 0.3:1 to 1.3:1. In determining said weight ratio, the following applies: a) said ethylene comprises unconverted ethylene that is recycled in step (e) to step (a); and b) said ethane comprises fresh ethane that is fed to step (a) and unconverted ethane that is recycled in step (e) to step (a). Said fresh ethane and said unconverted ethane and unconverted ethylene (i.e. recycle ethane and ethylene) may be fed via the same inlet or via two different inlets to a reactor used in step (a). Said weight ratio may be at least 0.1:1, preferably at least 0.2:1, more preferably at least 0.3:1, more preferably at least 0.4:1, more preferably at least 0.5:1, more preferably at least 0.6:1. Further, said weight ratio may be at most 2:1, preferably at most 1.8:1, more preferably at most 1.6:1, more preferably at most 1.5:1, more preferably at most 1.3:1, more preferably at most 1.1:1, more preferably at most 1:1, more preferably at most 0.9:1.

The conversion of ethane in step (a) may vary within wide ranges, and may be in the range of from 10 to 70%, suitably 15 to 60%.

Preferably, in ethane ODH step (a), that is to say during contacting ethylene and ethane with oxygen in the presence of a catalyst, the temperature is of from 300 to 500° C. More preferably, said temperature is of from 310 to 450° C., more preferably of from 320 to 420° C., most preferably of from 330 to 420° C.

Still further, in ethane ODH step (a), that is to say during contacting ethylene and ethane with oxygen in the presence of a catalyst, typical pressures are 1.1-30 or 1.1-20 or 1.1-15 bara (i.e. “bar absolute”). In the present invention, said pressure is preferably higher than 10 bara, more preferably higher than 10 bara up to 20 bara, most preferably of from 11 to 18 bara. Said pressure refers to total pressure.

One or more diluents, selected from the group consisting of the noble gases, nitrogen (N₂), steam (H₂O) and methane, may be fed to ethane ODH step (a), preferably steam and/or methane, most preferably methane. Some nitrogen and/or noble gases may be fed to step (a) as an impurity in the oxygen feed to step (a). In such case, they function as (additional) diluent. In case steam is fed as a diluent, it may be fed in a way as disclosed in WO2017198762, the disclosure of which is herein incorporated by reference. The use of methane as a diluent (in the present specification also referred to as “ballast gas”) in both step (a) and step (b) of the present process is further described below.

The oxygen as fed to ethane ODH step (a) is an oxidizing agent. Said oxygen may originate from any source, such as for example air. The molar ratio of oxygen to ethylene and ethane is suitably of from 0.01 to 1.1, more suitably of from 0.01 to 1, more suitably of from 0.05 to 0.8, more suitably of from 0.05 to 0.7, more suitably of from 0.1 to 0.6, more suitably of from 0.2 to 0.55, most suitably of from 0.25 to 0.5. Said ratio of oxygen to ethylene and ethane is the ratio before oxygen and ethylene and ethane are contacted with the catalyst. In other words, said ratio of oxygen to ethylene and ethane is the ratio of oxygen as fed to ethylene and ethane as fed. Obviously, after contact with the catalyst, at least part of the oxygen and ethylene and ethane gets consumed. Further, said “ethane” in said molar ratio of oxygen to ethylene and ethane comprises both fresh ethane and recycled (unconverted) ethane.

Preferably, pure or substantially pure oxygen (O₂) is used as oxidizing agent in step (a) of the process of the present invention. Within the present specification, by “pure or substantially pure oxygen” reference is made to oxygen that may contain a relatively small amount of one or more contaminants, including for example nitrogen (N₂) and/or argon, which latter amount may be at most 1 vol. %, suitably at most 7,000 parts per million by volume (ppmv), more suitably at most 5,000 ppmv, more suitably at most 3,000 ppmv, more suitably at most 1,000 ppmv, more suitably at most 500 ppmv, more suitably at most 300 ppmv, more suitably at most 200 ppmv, more suitably at most 100 ppmv, more suitably at most 50 ppmv, more suitably at most 30 ppmv, most suitably at most 10 ppmv.

Step (a) may be carried out in the presence of an ethane ODH catalyst, suitably in the presence of a catalyst comprising a mixed metal oxide. Preferably, the ODH catalyst is a heterogeneous catalyst. Further, preferably, the ODH catalyst is a mixed metal oxide catalyst containing molybdenum, vanadium, niobium and optionally tellurium as the metals, which catalyst may have the following formula:

Mo₁VₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for O) is a number which is determined by the valency and frequency of elements other than oxygen.

The amount of the catalyst in ethane ODH step (a) is not essential. Preferably, a catalytically effective amount of the catalyst is used, that is to say an amount sufficient to promote the desired reaction(s).

The ODH reactor that may be used in ethane ODH step (a) may be any reactor, including fixed-bed and fluidized-bed reactors. Suitably, the reactor is a fixed-bed reactor.

Examples of oxydehydrogenation processes, including catalysts and process conditions, are for example disclosed in above-mentioned U.S. Pat. No. 7,091,377, WO2003064035, US20040147393, WO2010096909 and US20100256432, the disclosures of which are herein incorporated by reference.

Step (b)

Step (b) of the present process comprises separating at least part of the stream resulting from step (a) into a stream comprising ethylene and ethane and a stream comprising water and acetic acid.

Step (b) may be carried out by condensation. Water and acetic acid in the stream resulting from step (a) may be condensed by cooling down the latter stream to a lower temperature, for example room temperature, after which the condensed water and acetic acid can be separated, resulting in a liquid stream comprising condensed water and acetic acid. During or after step (b), additional water may be added to facilitate the removal of acetic acid.

In step (b), the temperature may be of from 10 to 150° C., for example of from 20 to 80° C. Suitably, in said step (b), the temperature is at least 10° C. or at least 20° C. or at least 30° C. Further suitably, in said step (b), the temperature is at most 150° C. or at most 120° C. or at most 100° C. or at most 80° C. or at most 60° C.

Still further, in step (b), typical pressures are 1.1-30 or 1.1-20 bara (i.e. “bar absolute”). Further, preferably, said pressure is of from 1 to 18 bara, more preferably of from 3 to 16 bara, most preferably of from 5 to 15 bara. Said pressure refers to total pressure.

Thus, step (b) results in a stream comprising ethylene and ethane and a stream comprising water and acetic acid. The latter stream may be a liquid stream comprising condensed water and acetic acid.

Step (c)

Step (c) of the present process comprises producing ethylene oxide by subjecting ethylene and ethane from the stream comprising ethylene and ethane resulting from step (b) or from the below-mentioned additional carbon dioxide removal step between steps (b) and (c) to oxidation conditions, resulting in a stream comprising ethylene oxide, ethylene, ethane and water. Suitably, at least part of said stream is fed to step (c). It is preferred that ethylene and ethane from said stream are not separated from each other before feeding to step (c). Further, it is preferred that said stream is completely fed to step (c).

In addition, in step (c) of the present process, ethylene and ethane from the stream comprising ethylene and ethane resulting from step (d) or from the below-mentioned additional carbon dioxide removal step between steps (d) and (e) may be subjected to oxidation conditions. This is further described hereinbelow under “Step (e)”.

Ethylene oxide production step (c) may comprise contacting the ethylene and ethane with oxygen (O₂). Said oxygen as fed to step (c) is an oxidizing agent, and may be in the form of high-purity oxygen, preferably having a purity greater than 90%, preferably greater than 95%, more preferably greater than 99%, and most preferably greater than 99.4%. Suitable reaction pressures in ethylene oxide production step (c) are 1.1-30 bar, more suitably 3-25 bar, most suitably 5-20 bar. Suitable reaction temperatures in said step are 100-400° C., more suitably 200-300° C.

In the present invention, the weight ratio of ethylene to ethane as fed to ethylene oxide production step (c) may be in the range of from 0.1 to 10, preferably of from 0.3 to 8, more preferably of from 0.5 to 6. Said weight ratio may be at least 0.1, preferably at least 0.3, more preferably at least 0.5, more preferably at least 0.7, more preferably at least 1.0. Further, said weight ratio may be at most 10, preferably at most 8, more preferably at most 6, more preferably at most 5, more preferably at most 4.

Further, it is preferred that said contacting of ethylene and ethane with oxygen in step (c) is carried out in the presence of a catalyst, preferably a silver containing catalyst. A typical reactor for the ethylene oxide production step consists of an assembly of tubes that are packed with catalyst. A coolant may surround the reactor tubes, removing the reaction heat and permitting temperature control.

In case a silver containing catalyst is used in ethylene oxide production step (c), the silver in the silver containing catalyst is preferably in the form of silver oxide. Preferred is a catalyst comprising particles wherein silver is deposited on a carrier. Suitable carrier materials include refractory materials, such as alumina, magnesia, zirconia, silica and mixtures thereof. The catalyst may also contain a promoter component, e.g. rhenium, tungsten, molybdenum, chromium, nitrate- or nitrite-forming compounds and combinations thereof. Preferably, the catalyst is a pelletized catalyst, for example in the form of a fixed catalyst bed, or a powdered catalyst, for example in the form of a fluidized catalyst bed.

The nature of the ethylene oxidation catalyst, if any, is not essential in terms of obtaining the advantages of the present invention as described herein. The amount of the ethylene oxidation catalyst is neither essential. If a catalyst is used, preferably a catalytically effective amount of the catalyst is used, that is to say an amount sufficient to promote the ethylene oxidation reaction. Although a specific quantity of catalyst is not critical to the invention, preference may be expressed for use of the catalyst in such an amount that the gas hourly space velocity (GHSV) is of from 100 to 50,000 hr⁻¹, suitably of from 500 to 20,000 hr⁻¹, more suitably of from 1,000 to 10,000 hr⁻¹, most suitably of from 2,000 to 4,000 hr⁻¹.

In the present specification, “GHSV” or gas hourly space velocity is the unit volume of gas at normal temperature and pressure (0° C., 1 atmosphere, i.e. 101.3 kPa) passing over one unit volume of catalyst per hour.

Examples of ethylene oxidation processes, including catalysts and other process conditions, are for example disclosed in US20090281345 and GB1314613, the disclosures of which are herein incorporated by reference. All of these ethylene oxidation processes are suitable for ethylene oxidation step (c) of the present process.

Generally, a ballast gas (in the present specification also referred to as “diluent”) is added in an ethylene oxide production process. For in the oxidation of ethylene an oxidizing agent, such as high-purity oxygen, is required. Because an oxidizing agent is required, it is important to control the safe operability of the reaction mixture. Nitrogen, argon, methane or ethane may be utilized as such ballast gas. One function of a ballast gas is thus to control this safe operability.

In the present invention, both ethylene and ethane from the stream comprising ethylene and ethane resulting from step (b) are fed to ethylene oxide production step (c). Thus, unconverted ethane coming from ethane ODH step (a) may advantageously be used as a ballast gas in ethylene oxidation step (c) of the present process so that no or less additional ballast gas needs to be used. This results in a simpler and more efficient ethylene oxidation process as compared to a non-integrated process. If the amount of ethane in the stream resulting from step (b) is not sufficient, one or more additional gases selected from the group consisting of nitrogen, methane and ethane may be fed to step (c). Preferably, methane is fed as additional ballast gas. The use of methane as a diluent (in the present specification also referred to as “ballast gas”) in both step (a) and step (b) of the present process is further described below.

The amount of ethylene as fed to ethylene oxide production step (c) may be of from 1 to 50 wt. %, suitably of from 3 to 30 wt. %, more suitably of from 4 to 20 wt. %, most suitably of from 5 to 15 wt. %, based on total feed to step (c). The amount of ethane as fed to ethylene oxide production step (c) may be of from 1 to 50 wt. %, suitably of from 1 to 30 wt. %, more suitably of from 2 to 25 wt. %, most suitably of from 3 to 20 wt. %, based on total feed to step (c).

A moderator, for example a chlorohydrocarbon such as monochloroethane (ethyl chloride), vinyl chloride or dichloroethane, may be supplied for catalyst performance control in the ethylene oxide production step of the present process. Most suitably, ethyl chloride is used.

Moderators that can be suitably used in the ethylene oxide production step of the present process are also disclosed in above-mentioned GB1314613, the disclosure of which is herein incorporated by reference. GB1314613 discloses the use of an inhibitor (that is to say, a moderator), selected from ethylene dichloride, vinyl chloride, dichlorobenzene, monochlorobenzene, dichloromethane, and chlorinated phenyls, chlorinated biphenyls and chlorinated polyphenyls, in the production of ethylene oxide from ethylene.

The nature of the moderator, if any, is not essential in terms of obtaining the advantages of the present invention as described herein. The amount of such moderator in the reaction mixture may range from 1 part per million by volume (ppmv) to 2 vol. %, suitably 1 to 1,000 ppmv. The minimum amount of moderator in the reaction mixture may be 0.1 ppmv, 0.2 ppmv, 0.5 ppmv, 1 ppmv, 2 ppmv, 5 ppmv, 10 ppmv or 50 ppmv. The maximum amount of moderator in the reaction mixture may be 2 vol. %, 1 vol. %, 1,000 ppmv, 800 ppmv, 600 ppmv, 400 ppmv, 200 ppmv or 150 ppmv.

A suitable range for the amount of moderator that can be used in the ethylene oxide production step of the present process is also disclosed in above-mentioned GB1314613 in relation to the above-mentioned group of specific inhibitors (that is to say, moderators) as disclosed in said GB1314613, the disclosure of which is herein incorporated by reference.

Advantageously, any carbon monoxide and/or any acetylene present in the feed to step (c) does not need to be removed. For in ethylene oxidation step (c), carbon monoxide may be oxidized to carbon dioxide which in turn can be removed in accordance with the present invention, as further described below. Likewise, acetylene may be oxidized to carbon dioxide in said step (c). For example, the amount of acetylene in the feed to step (c) may be up to 1,000 parts per million by volume (ppmv), suitably at most 500 ppmv, more suitably at most 200 ppmv, based on total feed. Therefore, advantageously, by not having to remove any carbon monoxide and/or any acetylene an additional gas clean-up reactor may be omitted in the present process.

Alternatively, at least part of the feed to step (c) or at least part of the feed to below-mentioned additional carbon dioxide removal step between steps (b) and (c) may be subjected to a treatment wherein carbon monoxide and/or acetylene are oxidized into carbon dioxide. Preferably, said oxidation is performed in the presence of an oxidation catalyst, preferably an oxidation catalyst which comprises a transition metal. Preferably, said oxidation catalyst comprises one or more metals selected from the group consisting of nickel, copper, zinc, palladium, silver, platinum, gold, iron, manganese, cerium, tin, ruthenium and chromium. Further, preferably, said oxidation catalyst comprises copper and/or platinum, suitably copper or platinum, more suitably copper. The temperature during said oxidation treatment may be of from 50 to 500° C., for example of from 100 to 400° C. Preferably, said temperature is in the range of from 100 to 400° C., more preferably 150 to 300° C., most preferably 200 to 260° C.

The above-mentioned oxidation treatment may be carried out in a separate reactor upstream of step (c) or below-mentioned additional carbon dioxide removal step between steps (b) and (c). Alternatively, said treatment may be carried out inside a reactor used in step (c), namely in an upstream section thereof, wherein step (c) as such is carried out in a downstream section of said same reactor.

Step (d)

Step (d) of the present process comprises separating at least part of the stream comprising ethylene oxide, ethylene, ethane and water resulting from step (c) into a stream comprising ethylene and ethane and a stream comprising ethylene oxide and water.

Ethylene oxide can be recovered easily from the stream resulting from step (c) by means of methods known to the skilled person. Step (b) may be carried out in the same way as step (b), as described above, for example by condensation, taking into account the different boiling point for ethylene oxide to be recovered in step (d). The preferences and embodiments described for step (b) also apply to step (d).

Optional components that may also be present in the stream comprising ethylene oxide, ethylene, ethane and water resulting from step (c), which also comprises carbon dioxide, are: an additional ballast gas, a moderator and unconverted oxygen (O₂). Carbon dioxide is formed in step (c), and an additional ballast gas and a moderator may be used in step (c) as described above. Further, oxygen may be used as oxidizing agent in step (c). In such case, step (d) results in a stream comprising ethylene, ethane, carbon dioxide, optionally an additional ballast gas, optionally a moderator and optionally oxygen and a stream comprising ethylene oxide and water.

Step (e)

Step (e) of the present process comprises recycling ethylene and ethane from the stream comprising ethylene and ethane resulting from step (d) or from the below-mentioned additional carbon dioxide removal step between steps (d) and (e) to step (a). In the present process, it is preferred that ethylene from said stream is not recycled directly to step (c) or to below-mentioned additional carbon dioxide removal step between steps (b) and (c), but is only recycled indirectly via step (a). Suitably, at least part of said stream is recycled to step (a). It is preferred that ethylene and ethane from said stream are not separated from each other before recycling to step (a). Further, it is preferred that said stream is completely recycled to step (a).

In addition, in the present process, ethylene and ethane from the stream comprising ethylene and ethane resulting from step (d) or from the below-mentioned additional carbon dioxide removal step between steps (d) and (e) may be recycled to step (c) or to below-mentioned additional carbon dioxide removal step between steps (b) and (c).

In case there is no additional carbon dioxide removal step between steps (b) and (c), ethylene and ethane from the stream comprising ethylene and ethane resulting from the additional carbon dioxide removal step between steps (d) and (e) may be recycled to step (c). In said case, part of said stream is recycled to step (c). Further, in said case, it is preferred that ethylene and ethane from said stream are not separated from each other before recycling to step (c).

In case there is no additional carbon dioxide removal step between steps (d) and (e), ethylene and ethane from the stream comprising ethylene and ethane resulting from step (d) may be recycled to the additional carbon dioxide removal step between steps (b) and (c) or to step (c). In said case, part of said stream is recycled to the additional carbon dioxide removal step between steps (b) and (c) or to step (c). Further, in said case, it is preferred that ethylene and ethane from said stream are not separated from each other before recycling to the additional carbon dioxide removal step between steps (b) and (c) or to step (c).

In case there are both an additional carbon dioxide removal step between steps (b) and (c) and an additional carbon dioxide removal step between steps (d) and (e), ethylene and ethane from the stream comprising ethylene and ethane resulting from the additional carbon dioxide removal step between steps (d) and (e) may be recycled to step (c). In said case, part of said stream is recycled to step (c). Further, in said case, it is preferred that ethylene and ethane from said stream are not separated from each other before recycling to step (c).

Recycling part of the stream comprising ethylene and ethane resulting from step (d) or from the below-mentioned additional carbon dioxide removal step between steps (d) and (e) to step (c) or to below-mentioned additional carbon dioxide removal step between steps (b) and (c), as described above, may be performed by splitting said stream into streams (i) and (ii), wherein stream (i) is recycled to step (a) and stream (ii) is recycled to step (c) or to an additional carbon dioxide removal step between steps (b) and (c).

Together with recycling ethylene and ethane in step (e), as described above, a moderator as used in step (c) as described above may be present in a stream comprising ethylene and ethane that is recycled in step (e). The amount of moderator in said stream may range from 1 part per million by volume (ppmv) to 2 vol. %, suitably 1 to 1,000 ppmv. The minimum amount of moderator in said stream may be 0.1 ppmv, 0.2 ppmv, 0.5 ppmv, 1 ppmv, 2 ppmv, 5 ppmv, 10 ppmv or 50 ppmv. The maximum amount of moderator in said stream may be 2 vol. %, 1 vol. %, 1,000 ppmv, 800 ppmv, 600 ppmv, 400 ppmv, 200 ppmv or 150 ppmv.

Carbon Dioxide Removal

In the present invention, carbon dioxide is produced in ethane ODH step (a) and in ethylene oxide production step (c). Further, in the present invention, said carbon dioxide is removed in an additional step between steps (b) and (c) and/or between steps (d) and (e). In the present invention, there may be one carbon dioxide removal step, namely between steps (b) and (c) or between steps (d) and (e). Further, in the present invention, there may be two carbon dioxide removal steps, namely between steps (b) and (c) and between steps (d) and (e). In the present invention, it is preferred that there is a carbon dioxide removal step between steps (b) and (c). More preferably, in the present invention, there is one carbon dioxide removal step and said step is between steps (b) and (c). Thus, in said more preferred case, there is no carbon dioxide removal step between steps (d) and (e).

In a case where in the present invention carbon dioxide is removed in an additional step between steps (b) and (c), the stream resulting from step (a) comprises ethylene, ethane, water, acetic acid and carbon dioxide, at least part of which stream is separated in step (b) into a stream comprising ethylene, ethane and carbon dioxide and a stream comprising water and acetic acid. Further, said additional step between steps (b) and (c) comprises removing carbon dioxide from at least part of the stream comprising ethylene, ethane and carbon dioxide resulting from step (b), resulting in a stream comprising ethylene and ethane. Further, in step (c), ethylene oxide is produced by subjecting ethylene and ethane from the latter stream comprising ethylene and ethane to oxidation conditions.

In the ethane ODH embodiment as shown in FIG. 3 of above-mentioned WO2012101069, no carbon dioxide is removed between the ethane ODH and ethylene oxide production steps, so that carbon dioxide may be sent to the ethylene oxide production step. In the above-mentioned additional carbon dioxide removal step between steps (b) and (c) in the present invention, it is advantageously prevented that carbon dioxide is fed to ethylene oxide production in step (c). It is well-known that the presence of carbon dioxide during ethylene oxide production, as in step (c) of the present process, reduces the activity and/or selectivity (towards ethylene oxide) of the catalyst used in such step.

In a case where in the present invention carbon dioxide is removed in an additional step between steps (d) and (e), the stream resulting from step (c) comprises ethylene oxide, ethylene, ethane, water and carbon dioxide, at least part of which stream is separated in step (d) into a stream comprising ethylene, ethane and carbon dioxide and a stream comprising ethylene oxide and water. Further, said additional step between steps (d) and (e) comprises removing carbon dioxide from at least part of the stream comprising ethylene, ethane and carbon dioxide resulting from step (d), resulting in a stream comprising ethylene and ethane. Further, in step (e), ethylene and ethane from the latter stream comprising ethylene and ethane is recycled to step (a).

In the above-mentioned additional carbon dioxide removal step or steps, carbon dioxide may be removed by any one of well-known methods. A suitable carbon dioxide removal agent that may be fed to such step may be an aqueous solution of a base, for example sodium hydroxide and/or an amine. After such carbon dioxide removal, the stream from which carbon dioxide is removed may be dried to remove any residual water from the stream before it is fed to the next step.

Methane as Diluent in Steps (a) and (c)

Most preferably, in the present invention, methane is used as a diluent in both step (a) and step (c), and methane from the stream resulting from step (c) is recycled to step (a).

In the above-mentioned case, the present process comprises the steps of:

(a) producing ethylene by subjecting a stream comprising ethane, ethylene and methane to oxidative dehydrogenation conditions, resulting in a stream comprising ethylene, ethane, methane, water and acetic acid;

(b) separating at least part of the stream resulting from step (a) into a stream comprising ethylene, ethane and methane and a stream comprising water and acetic acid;

(c) producing ethylene oxide by subjecting ethylene, ethane and methane from the stream comprising ethylene, ethane and methane resulting from step (b) to oxidation conditions, resulting in a stream comprising ethylene oxide, ethylene, ethane, methane and water;

(d) separating at least part of the stream resulting from step (c) into a stream comprising ethylene, ethane and methane and a stream comprising ethylene oxide and water;

(e) recycling ethylene, ethane and methane from the stream comprising ethylene, ethane and methane resulting from step (d) to step (a),

wherein carbon dioxide is produced in steps (a) and (c) and is removed in an additional step between steps (b) and (c) and/or between steps (d) and (e).

Further, in the above-mentioned case, some methane may be lost during separation steps (b) and (d) and the carbon dioxide removal step(s). In such a case, the relative amount of methane may be kept constant by feeding a make-up stream comprising methane to the present process. Such make-up stream may be fed to step (a) and/or step (c), preferably step (c).

Advantages of the Present Invention

Thus, in the present invention, there is advantageously no need at all to separate ethane from ethylene before recycling to step (a), as both ethylene and ethane are recycled to step (a). On the contrary, in the ethane ODH embodiment of the process of above-discussed WO2012101069 ethylene and ethane have to be separated, thereby enabling a direct recycle of ethylene to the ethylene oxidation step whereas ethane is recycled to the ethane ODH step. Such additional ethylene and ethane separation step, which is advantageously avoided in the present process, is cumbersome as it requires the use of an ethylene/ethane splitter wherein ethylene and ethane are separated by means of cryogenic distillation resulting in a high energy and capital expenditure.

Furthermore, in step (e) of the present process, at least part of a stream comprising ethylene, ethane, carbon dioxide, optionally an additional ballast gas, optionally a moderator and optionally oxygen resulting from step (d) may be recycled to step (a), that is to say without any intermediate separation/treatment steps between said steps (d) and (a). Apart from not having to separate ethane and ethylene as discussed above, this results in the following additional advantages.

It is advantageous not having to remove unconverted oxygen from the above-mentioned stream before recycling to step (a) as in step (a) such oxygen may still be used as an oxidizing agent as described above. Furthermore, since in the present invention no cryogenic distillation needs to be applied to separate ethane and ethylene, the safety risk caused by the presence of oxygen in cryogenic distillation cannot occur. Such risk could be reduced by a cumbersome removal of unconverted oxygen upstream of said cryogenic distillation step. Thus, in the present process, advantageously, there is no such safety risk and consequently there is neither any need for above-described oxygen removal.

In addition, in the present invention, there is advantageously no need to directly feed oxygen to step (a), but oxygen can be fed indirectly via feeding to step (c) and recycling in step (e) to step (a). Therefore, in the present invention there only needs to be one oxygen feed point, namely for ethylene oxidation step (c), especially in a case wherein oxygen conversion in said step (c) is kept relatively low. Such single oxygen feed point may be placed anywhere between steps (a) and (c), but preferably oxygen from said oxygen feed point is directly fed into step (c). Having a single oxygen feed point in an integrated process is advantageous as it reduces the oxygen handling risk and saves on equipment costs.

The foregoing is different from the ethane ODH embodiment as shown in FIG. 3 of above-mentioned WO2012101069 wherein oxygen is fed separately to ethane ODH and ethylene oxide production via two different feed points, in streams 15 and 4, respectively. Part of the oxygen that is not converted in ethylene oxide production in the process of said FIG. 3 is recycled back to the ethylene oxide production unit via streams 9a and 11a. The rest of the unconverted oxygen is fed to ethylene/ethane separation unit 12 wherein the oxygen is purged as incondensable gas in a top bleed stream (which may be a safety risk). This implies that no oxygen is present in the recycle to the ethane ODH unit, which makes that an additional oxygen feed point for the ethane ODH unit is required. The loss of oxygen feedstock in a purge stream and the requirement of an additional feed point for oxygen, as described above, are advantageously avoided in the present invention. Since in the present invention, there is no such loss of oxygen feedstock, neither is there any need to maximize the conversion of oxygen in step (c) to reduce said oxygen loss, which relatively high conversion would disadvantageously result in a decrease of selectivity towards ethylene oxide.

Further, it is advantageous not having to remove carbon dioxide from the above-mentioned stream comprising ethylene, ethane, carbon dioxide, optionally an additional ballast gas, optionally a moderator and optionally oxygen resulting from step (d), before recycling to step (a). Furthermore, carbon dioxide as formed in both ethane ODH and ethylene oxide production may still be removed jointly in a single additional carbon dioxide removal step between steps (b) and (c), thereby at the same time also avoiding a negative effect of carbon dioxide on the ethylene oxidation reaction, as described above.

Still further, it is advantageous not having to remove an additional ballast gas (e.g. methane) from the above-mentioned stream before recycling to step (a) as in step (a) such additional ballast gas may still be used as a diluent as described above. A relatively high amount of such additional ballast gas in ethane ODH step (a) further helps in keeping the oxygen concentration in the ethane ODH reactor low, thereby advantageously reducing flammability risks.

Finally, as mentioned above, in a case where in the present invention a moderator is used in the ethylene oxide production step, such moderator is also recycled to step (a). Surprisingly, it has been found that such moderator as recycled to step (a) advantageously does not interfere with the ethane ODH reaction in step (a).

Therefore, in the present process, any intermediate separation/treatment steps are not required after having removed ethylene oxide product and water in step (d) and before recycling in step (e). Avoiding above-mentioned ethane/ethylene separation is a significant advantage since it results in a much simpler process using less separation processes and equipment as well as a substantial reduction of expenditure, for example savings on costs for compression, refrigeration, etc.

Further, surprisingly, the ethylene that is recycled in the present process to ethane ODH step (a) is advantageously mainly converted into acetic acid (and to a lesser extent into carbon oxides), which is another valuable product, in addition to ethylene oxide. Above-mentioned WO2012101069 is focused on the production of ethylene oxide only, and does not disclose the co-production of ethylene oxide and acetic acid in one integrated process. Furthermore, acetic acid is easily recovered in water removal step (b) of the present process. This is not an additional step as in WO2012101069 such water removal step is also applied.

Overall, in the present integrated process, a single loop may advantageously be established by feeding additional ballast gas (make-up stream) and oxygen at only one point in the overall process, preferably in the feed to the ethylene oxide production step, and feeding fresh ethane to the ethane ODH step, and recycling the effluent from said ethylene oxide production step, after having only removed ethylene oxide and water therefrom and not having to separate ethane and ethylene and any other components, completely to ethane ODH step (a), in which step (a) the additional ballast gas and (unconverted) oxygen may still be used and valuable acetic acid is formed as a valuable co-product, and carbon dioxide formed in reaction steps (a) and (c) may still be removed jointly in a single step between steps (b) and (c), without the moderator having a negative effect on the ethane ODH reaction in step (a).

Monoethylene Glycol Production

Preferably, at least part of the ethylene oxide is converted to monoethylene glycol (MEG), which is a useful liquid product. Thus, the present invention also relates to a process for the production of monoethylene glycol, comprising the steps of: producing ethylene oxide by the present process as described above; and converting at least part of the ethylene oxide to monoethylene glycol.

The conversion of ethylene oxide to MEG may be done using any MEG producing process that uses ethylene oxide. Typically the ethylene oxide is hydrolysed with water to MEG. Optionally, the ethylene oxide is first converted with carbon dioxide to ethylene carbonate, which is subsequently hydrolysed to MEG and carbon dioxide. The water is provided to the MEG zone as a feed containing water, preferably pure water or steam. The MEG product is obtained from the MEG zone as a MEG-comprising effluent. Suitable processes for the production of ethylene oxide and MEG are described for instance in US2008139853, US2009234144, US2004225138, US20044224841 and US2008182999, the disclosures of which are herein incorporated by reference.

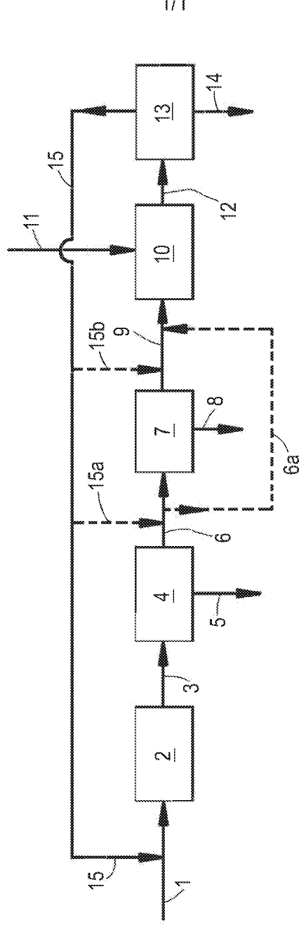

The invention is further illustrated in FIG. 1 as described hereinbelow.

FIG. 1

In the flow scheme of FIG. 1, stream 1 comprising fresh ethane is fed to ethane ODH unit 2. Recycle stream 15 comprising ethylene, ethane, methane, carbon dioxide and oxygen is also fed to ethane ODH unit 2. Stream 3 comprising ethylene, ethane, methane, water, acetic acid and carbon dioxide coming from ethane ODH unit 2 is sent to water removal unit 4, wherein water and acetic acid are removed via stream 5. Stream 6 comprising ethylene, ethane, methane and carbon dioxide coming from water removal unit 4, optionally combined with below-mentioned substream 15a, is sent to carbon dioxide removal unit 7 wherein carbon dioxide is removed via stream 8. Optionally, stream 6 optionally combined with below-mentioned substream 15a is split and substream 6a is fed directly to ethylene oxide production unit 10.

Stream 9 comprising ethylene, ethane and methane coming from carbon dioxide removal unit 7, optionally combined with below-mentioned substream 15b, and stream 11 comprising oxygen are fed to ethylene oxide production unit 10. Further, a make-up stream comprising methane (not shown in FIG. 1) is fed to ethylene oxide production unit 10. Stream 12 comprising ethylene oxide, ethylene, ethane, methane, carbon dioxide, water and oxygen coming from ethylene oxide production unit 10 is sent to ethylene oxide separation unit 13. Ethylene oxide and water are recovered via stream 14. Further, stream 15 comprising ethylene, ethane, methane, carbon dioxide and oxygen is recycled to ethane ODH unit 2. Optionally, stream 15 is split and substream 15a and/or substream 15b is/are recycled to carbon dioxide removal unit 7 and ethylene oxide production unit 10, respectively.

1. A process for the production of ethylene oxide, comprising the steps of:
(a) producing ethylene by subjecting a stream comprising ethane to oxidative dehydrogenation conditions, resulting in a stream comprising ethylene, ethane, water and acetic acid;
(b) separating at least part of the stream resulting from step (a) into a stream comprising ethylene and ethane and a stream comprising water and acetic acid;
(c) producing ethylene oxide by subjecting ethylene and ethane from the stream comprising ethylene and ethane resulting from step (b) to oxidation conditions, resulting in a stream comprising ethylene oxide, ethylene, ethane and water;
(d) separating at least part of the stream resulting from step (c) into a stream comprising ethylene and ethane and a stream comprising ethylene oxide and water;
(e) recycling ethylene and ethane from the stream comprising ethylene and ethane resulting from step (d) to step (a),
wherein carbon dioxide is produced in steps (a) and (c) and is removed in an additional step between steps (b) and (c) and/or between steps (d) and (e).

2. The process according to claim 1, wherein the weight ratio of ethylene to ethane as fed to step (a) is in the range of from 0.1:1 to 2:1, preferably of from 0.2:1 to 1.5:1, more preferably of from 0.3:1 to 1.3:1.

3. The process according to claim 1, wherein the weight ratio of ethylene to ethane as fed to step (c) is in the range of from 0.1 to 10, preferably of from 0.3 to 8, more preferably of from 0.5 to 6.

4. The process according to claim 1, wherein at least part of the stream comprising ethylene and ethane resulting from step (d) is recycled to step (a).

5. The process according to claim 4, wherein the stream comprising ethylene and ethane resulting from step (d) is completely recycled to step (a).

6. The process according to claim 1, wherein in step (c) a moderator is used and wherein moderator is present in a stream comprising ethylene and ethane that is recycled in step (e), preferably in an amount of from 1 part per million by volume (ppmv) to 2 vol. %, more preferably 1 to 1,000 ppmv.

7. The process according to claim 1, wherein there is a carbon dioxide removal step between steps (b) and (c), preferably wherein there is one carbon dioxide removal step and said step is between steps (b) and (c).

8. The process according to claim 1, wherein methane is used as a diluent in both step (a) and step (c), and methane from the stream resulting from step (c) is recycled to step (a).

9. The process according to claim 8, which comprises the steps of:
(a) producing ethylene by subjecting a stream comprising ethane, ethylene and methane to oxidative dehydrogenation conditions, resulting in a stream comprising ethylene, ethane, methane, water and acetic acid;
(b) separating at least part of the stream resulting from step (a) into a stream comprising ethylene, ethane and methane and a stream comprising water and acetic acid;
(c) producing ethylene oxide by subjecting ethylene, ethane and methane from the stream comprising ethylene, ethane and methane resulting from step (b) to oxidation conditions, resulting in a stream comprising ethylene oxide, ethylene, ethane, methane and water;
(d) separating at least part of the stream resulting from step (c) into a stream comprising ethylene, ethane and methane and a stream comprising ethylene oxide and water;
(e) recycling ethylene, ethane and methane from the stream comprising ethylene, ethane and methane resulting from step (d) to step (a),
wherein carbon dioxide is produced in steps (a) and (c) and is removed in an additional step between steps (b) and (c) and/or between steps (d) and (e).

10. A process for the production of monoethylene glycol, comprising the steps of:
producing ethylene oxide by the process according to claim 1; and
converting at least part of the ethylene oxide to monoethylene glycol.
